(19) United States
(12) Patent Application Publication
(10) Pub. No.: US 2015/0329882 A1
(43) Pub. Date: Nov. 19, 2015

(54) MICROORGANISM HAVING ENHANCED PRODUCTIVITY OF SUCCINATE AND METHOD OF PRODUCING SUCCINATE USING THE SAME

(71) Applicant: Samsung Electronics Co., Ltd., Suwon-si (KR)

(72) Inventors: Wooyong Lee, Hwaseong-si (KR); Jinhwan Park, Suwon-si (KR); Kwangmyung Cho, Seongnam-si (KR)

(21) Appl. No.: 14/715,203

(22) Filed: May 18, 2015

(30) Foreign Application Priority Data
May 16, 2014 (KR) 10-2014-0059300

(51) Int. Cl. C12P 7/46; C12N 9/02; C12N 15/77

(52) U.S. Cl. CPC C12P 7/46; C12N 15/77; C12N 9/0008; C12Y 102/04001; C12Y 102/04002

(57) ABSTRACT

Provided is a recombinant microorganism comprising an exogenous pyruvate dehydrogenase E1 protein, and/or increased expression of alpha-ketoglutarate dehydrogenase E1.

### RELATED APPLICATION

This application claims the benefit of Korean Patent Application No. 10-2014-0059300, filed on May 16, 2014, in the Korean Intellectual Property Office, the entire disclosure of which is hereby incorporated by reference.

### INCORPORATION-BY-REFERENCE OF MATERIAL ELECTRONICALLY SUBMITTED

Incorporated by reference in its entirety herein is a computer-readable nucleotide/amino acid sequence listing submitted herewith and identified as follows: One 87,057 byte ASCII (Text) file named “719797_ST25.TXT,” created May 1, 2015.

### BACKGROUND

1. Field

The present disclosure relates to microorganisms having enhanced capability of producing succinate, and a method of producing succinate by using the same.

2. Description of the Related Art

Corynebacterium is a genus of gram-positive and has been widely used for the production of amino acids, such as glutamate, lysine, and threonine. C. glutamicum has advantages as an industrial strain due to it requiring simple growth conditions, its stable genome structure, and because it does not present environmental hazards.

C. glutamicum is an aerobic bacterium so that metabolic processes thereof, except those processes that generate the minimum amount of energy for the survival, stop under anaerobic conditions. Under anaerobic conditions, in order to generate energy, C. glutamicum releases lactate, acetate, and succinate.

As an intermediate product produced in a tricarboxylic acid (TCA) cycle, succinate is the simplest dicarboxylic organic acid and may be used to produce various polyester compounds.

Because of increasing demands for succinate, there has been an increased demand for methods of efficiently producing succinate by using a microorganism.

### SUMMARY

Provided is a recombinant microorganism comprising (a) an exogenous pyruvate dehydrogenase E1, (b) increased expression of alpha-ketoglutarate dehydrogenase E1 compared to a reference microorganism, or both (a) and (b).

Further provided is a method of increasing succinate production in a micoorgansim by (a) introducing an exogenous polynucleotide encoding pyruvate dehydrogenase E1 into the microorganism; (b) increasing the expression of alpha-ketoglutarate dehydrogenase E1 in the microorganism, or both (a) and (b).

Also provided is a method of producing succinate by using the recombinant microorganisms.

Additional aspects will be set forth in part in the description which follows and, in part, will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1 is a vector map of a vector pGSK+;

FIG. 2 is a vector map of a vector pGS-Term;

FIG. 3 is a vector map of a vector pGS-Ex2 EGFP-H6;

FIG. 4 is a graph showing yields of succinate under culturing conditions of 4HB, m4HB, Saline, CB, CB+CC, CGXII, and CGXII/0oxy;

FIG. 5 is a graph showing a correlation between glucose consumption and ultimate pH;

FIG. 6 is a graph showing screening results of succinate productivity and indicating major genes; and

FIG. 7 is a graph showing results of evaluation of succinate productivity in a CGXII medium and in a fermentation broth.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items.

Provided are recombinant microorganisms having an exogenous pyruvate dehydrogenase E1 (e.g., comprising an exogenous polynucleotide encoding pyruvate dehydrogenase E1); having an increased expression of alpha-ketoglutarate dehydrogenase E1; or a combination thereof.

The microorganism may include Archaebacterium, Eubacterium, or a eukaryotic microorganism, such as yeast and fungi. The microorganism may belong to the genus Corynebacterium and may be C. glutamicum.

The term “exogenous” polypeptide or polynucleotide refers to a polypeptide or a polynucleotide that originated outside of a host cell and is introduced to a host cell. Such introduction may involve, for example, integration to the genome of the host cell. The introduced polypeptide or polynucleotide may be homologous or heterologous with respect to the host cell. As used herein, the term “homologous” refers to a polypeptide or polynucleotide derived from a species identical to a host cell, whereas the term “heterologous” refers to a polypeptide or polynucleotide derived from a different species from a host cell.

The pyruvate dehydrogenase E1 may be naturally present as one of three enzymatic components of a pyruvate dehydrogenase complex (PDC) that catalyzes conversion of pyruvate into acetyl-CoA, wherein the PDC additionally includes a dihydrolipoyl transacetylase component (E2) and a dihydrolipoyl dehydrogenase component (E3). The pyruvate dehydrogenase E1 may be an enzyme categorized as EC.1.2.4.1. In addition, the pyruvate dehydrogenase E1 may use thiamine pyrophosphate (TPP) as a cofactor.

The exogenous pyruvate dehydrogenase E1 may have an amino acid sequence of SEQ ID NO: 1, and may be derived from the genus Escherichia, the genus Shigella, or the genus Salmonella. For example, the exogenous pyruvate dehydrogenase E1 may be derived from E. coli.

In an exemplary embodiment, the microorganism may be a microorganism belonging to the genus Corynebacterium and including a polynucleotide that encodes SEQ ID NO: 1. The microorganism may include a polynucleotide that is operably linked with a regulatory sequence and that encodes SEQ ID NO: 1. The term “operably linked” used herein refers to a functional linkage between a gene to be expressed and a regulatory sequence of the gene to enable gene expression. The regulatory sequence may include a promoter, an enhancer, a terminator, or a combination thereof.

The alpha-ketoglutarate dehydrogenase E1 may be naturally present as a subunit of an α-ketoglutarate dehydrogenase complex (α-KGDH complex) that catalyzes conversion of α-ketoglutarate into succinyl-CoA, wherein the α-KGDH complex additionally includes a transsuccinylase component (E2) and a dihydrolipoyl dehydrogenase component (E3). The α-KGDH complex may be an enzyme catalyzed as EC.1.2.4.2. In addition, the α-KGDH complex may also be referred to as an oxoglutarate dehydrogenase complex.

The increased expression of the α-ketoglutarate dehydrogenase E1 may be achieved by an increased expression of a polynucleotide that encodes the E1 compared to a reference microorganism. The increased expression of the polynucleotide may be caused by mutation in a regulatory region thereof. Alternatively, or in addition, the increased expression of the α-ketoglutarate dehydrogenase E1 may be achieved by introduction of an exogenous polynucleotide that encodes the α-ketoglutarate dehydrogenase E1 into the microorganism. The polynucleotide may belong to the genus Corynebacterium. The polynucleotide may be a nucleotide that encodes an SEQ ID NO: 2, and may comprise SEQ ID NO: 3.

In the microorganism, expression of phosphoenolpyruvate carboxlyase, phosphoenolpyruvate carboxykinase, alpha-ketoglutarate decarboxlyase, or a combination thereof may be increased compared to a reference microorganism.

The phosphoenolpyruvate carboxlyase (PEPC) may catalyze a reaction by which bicarbonate is added to phosphoenolpyruvate to form oxaloacetate. The PEPC may be an enzyme categorized as EC.4.1.1.31, and may comprise SEQ ID NO: 4. The increased expression of the PEPC may be achieved by an increased expression of a polynucleotide that encodes the PEPC compared to a reference microorganism or by introduction of a polynucleotide that encodes the PEPC into the recombinant microorganism.

The phosphoenolpyruvate carboxykinase (PEPCK) may catalyze a reaction by which oxaloacetate is converted into phosphoenolpyruvate. The PEPCK may be an enzyme categorized as EC.4.1.1.32, and may comprise SEQ ID NO: 5. The increased expression of the PEPCK may be achieved by an increased expression of a polynucleotide that encodes the PEPCK compared to a reference microorganism or by introduction of a polynucleotide that encodes the PEPCK into the recombinant microorganism.

The alpha-ketoglutarate decarboxylase (AKDC) may catalyze a reaction by which alpha-ketoglutarate is converted into succinic semialdehyde. The AKDC may be an enzyme categorized as EC.4.1.1.71, and may comprise SEQ ID NO: 6. The increased expression of the AKDC may be achieved by an increased expression of a polynucleotide that encodes the AKDC compared to a reference microorganism or by introduction of a polynucleotide that encodes the AKDC into the recombinant microorganism.

In the microorganism, activities of catalyzing conversion of pyruvate into lactate, acetyl CoA into acetate, or a combination thereof may be removed or reduced. The term “reduction” as used herein indicates a decreased activity (e.g., decreased enzymatic activity) in the recombinant microorganism compared to a reference microorganism. The term “reference microorganism” as used herein refers to a wild-type microorganism or a parental microorganism, e.g., such as a microorganism that does not comprise one or more particular genetic modifications that produce the recombinant microorganism (e.g., an exogenous polypeptide, an exogenous polynucleotide, and/or other genetic modifications). By way of further example, the parental microorganism refers to a microorganism that has not undergone one or more modifications that are present in the recombinant microorganism, and is thus genetically identical except for said modification(s) and thus serves as a reference microorganism for the modification.

The microorganism in which the activities of catalyzing conversion of pyruvate into lactate, acetyl CoA into acetate, or a combination thereof are removed or reduced may have a polynucleotide that encodes L-lactate dehydrogenase, a polynucleotide that encodes pyruvate oxidase, a polynucleotide that encodes phosphotransacetylase, a polynucleotide that encodes acetate kinase, a polynucleotide that encodes acetate coenzyme A transferase, or a combination thereof, wherein activities of these polynucleotides are inactivated or attenuated compared to a reference microorganism.

The term “inactivation” as used herein refers to a modification to a gene that results in the termination of the gene's expression or whereby the expression of the gene does not result in any corresponding enzymatic activity. The term “attenuation” as used herein refers a modification to a gene that results in a reduced expression level compared to a reference microorganism. The inactivation or attenuation in the microorganism may be caused by, for example, a homologous recombination. The inactivation or attenuation in the microorganism may be achieved by, for example, transforming a cell to which a vector including a partial nucleotide sequence of a gene to be recombinated, culturing the cell, inducing a homologous recombination between the partial nucleotide sequence and a sequence of an endogenous gene of the cell, and selecting a cell by using a selective marker in which the homologous recombination is occurred.

The lactate dehydrogenase may be an enzyme categorized as EC.1.1.1.27, and may have an amino acid sequence of SEQ ID NO: 7. The pyruvate oxidase may be an enzyme categorized as EC.1.2.3.3, and may have an amino acid sequence of SEQ ID NO: 8. The phosphotransacetylase may be an enzyme categorized as EC.2.3.1.8, and may have an amino acid sequence of SEQ ID NO: 9. The acetate kinase may be an enzyme categorized as EC.2.7.2.1, and may have an amino acid sequence of SEQ ID NO: 10. The acetate coenzyme A transferase may be an enzyme categorized as EC.2.8.3.8, and may have an amino acid sequence of SEQ ID NO: 11.

The microorganism may have an increased activity of pyruvate carboxylase (PC) that catalyzes conversion of pyruvate into oxaloacetate compared to a reference microorganism. The PC may be an enzyme categorized as EC.6.4.1.1, and may have an amino acid sequence of SEQ ID NO: 12. The increased expression of the PC may be achieved by an increased expression of a polynucleotide that encodes the PC compared to a reference microorganism or by introduction of a polynucleotide that encodes the PC into the recombinant microorganism.

In addition, the increased activity of the microorganism may be achieved by introduction of a polynucleotide that encodes the PC having an increased specific activity due to a mutation into the recombinant microorganism. The mutation may include substitution, addition, deletion, or a combination of the amino acid sequence of PC. One such substitution in the amino acid sequence occurs by the substitution of proline at the 458^{th} position in the amino acid sequence of SEQ ID NO: 12 with serine. The increased activity of the microorganism may be caused by genetic engineering.

Also provided herein is a method increasing succinate production in a micoorgansim by (a) introducing an exogenous pyruvate dehydrogenase E1 gene into the microorganism; (b) increasing the expression of alpha-ketoglutarate dehydrogenase E1 in the microorganism, or both (a) and (b). All other aspects of the method are as described in connection with the recombinant microorganism.

Provided are methods of producing succinate, the method including: culturing the recombinant microorganism in a cell culture medium, whereby the microorganism produces succinate; and collecting succinate from the cultured microorganism.

The culturing may be vary according to suitable media and culturing conditions known in the art, and one of ordinary skill in the art may be able to regulate media and culturing conditions according to a selected microorganism. The culturing may include a batch culture, a continuous culture, a fed-batch culture, or a combination thereof.

The medium used herein may include various carbon sources, nitrogen sources, and trace element components. The carbon sources may be, for example, carbohydrates including glucose, sucrose, lactose, fructose, maltose, starch, and cellulose, fats including soybean oil, sunflower oil, castor oil, and coconut oil, fatty acids including palmitic acid, stearic acid, and linoleic acid, alcohol including glycerol and ethanol, organic acids including acetic acid, or a combination thereof. The culturing may be performed by using glucose as a carbon source. The nitrogen sources may be, for example, organic nitrogen sources including peptone, yeast extract, meat extract, malt extract, corn steep liquor (CSL), and soybean wheat, and inorganic nitrogen sources including urea, ammonium sulfate, ammonium chloride, ammonium phosphate, ammonium carbonate, and ammonium nitrate, or a combination thereof. The medium used herein may use phosphorous sources, such as potassium dihydrogen phosphate, dipotassium hydrogen phosphate, sodium-containing salts corresponding thereto, or metal salts including magnesium sulfate or iron sulfate. In addition, amino acids, vitamins, and appropriate precursors may be contained in the medium. The medium or individual components may be added to a culture broth in the form of a batch culture or a continuous culture.

In addition, during culturing, compounds, such as ammonium hydroxide, potassium hydroxide, ammonia, phosphoric acids, and sulfuric acids, may be added in an appropriate manner into a microbial culture broth, thereby adjusting pH of the microbial culture broth. Furthermore, in the middle of culturing, an anti-foaming agent such as fatty acid polyglycol ester may be used to inhibit generation of foams.

The culturing may be performed in aerobic, microaerobic, or anaerobic conditions. As used herein, the term “microaerobic conditions when used in reference to a culture or growth condition indicates that the dissolved oxygen concentration in the medium remains between about 0% and about 10% of saturation for dissolved oxygen in liquid media. Microaerobic conditions also include growing or resting cells in liquid medium or on solid agar inside a sealed chamber maintained with an atmosphere of less than 1% oxygen. The percent of oxygen can be maintained by, for example, sparging the culture with an N₂/CO₂ mixture or other suitable non-oxygen gas or gases. As used herein, the term “anaerobic conditions” refers to an environment devoid of oxygen. In an exemplary embodiment, the culturing may be performed in an anaerobic condition. The anaerobic condition may be prepared by, for example, an incubator capable of adjusting oxygen concentration in atmosphere. Suitable oxygen concentration in an anaerobic condition may range between about 1% and about 10%, about 1.5% and about 8%, about 1.8% and about 6%, about 2% and about 4%, or about 2.5% and about 3%. The culturing temperature may range, for example between about 20° C. and about 45° C. or between about 25° C. and about 40° C.

The collecting of succinate may be performed according to separation and purification methods known in the art, for example, centrifugation, ion-exchange chromatography, filtration, precipitation, or a combination thereof.

Hereinafter, the present invention is described in greater detail with reference to embodiments. However, the embodiments are for illustrative purposes only and do not limit the scope of the present invention.

### EXAMPLE 1

### Preparation of a Host Strain for Preparing a Gene Expression Library

(1) Preparation of a Replacement Vector

L-lactate dehydrogenase (ldh), pyruvate oxidase (poxB), phosphotransacetylase (pta), acetate kinase (ackA), and acetate coenzyme A-transferase (actA) genes of C. glutamicum (CGL) ATCC 13032 were inactivated by a homologous recombination. Here, a vector pK19 mobsacB (ATCC 87098) was used to inactivate these genes, and two homologous regions to be used for a recombination were obtained by PCR amplification using genomic DNA of CGL ATCC 13032 as a template.

The two homologous regions for removing ldh gene are upstream and downstream regions of the gene and were obtained by PCR amplification by using a primer set of ldhA_{—}5′_HindIII (SEQ ID NO: 13) and ldhA_up_{—}3′_XhoI (SEQ ID NO: 14) and a primer set of ldhA_dn_{—}5′_XhoI (SEQ ID NO: 15) and ldhA_{—}3′_EcoRI (SEQ ID NO: 16), respectively. The PCR amplification was performed by repeating a cycle 30 times, wherein the cycle consisted of denaturation at a temperature of 95° C. for 30 seconds, annealing at a temperature of 55° C. for 30 seconds, and elongation at a temperature of 72° C. for 30 seconds. Hereinafter, all the PCR amplifications were performed in the same manner. Products obtained from the PCR amplification were cloned between restriction enzyme sites of HindIII and EcoRI, in the vector pK19 mobsacB, thereby manufacturing a vector pK19_Δldh.

The two homologous regions for removing poxB gene is upstream and downstream regions of the gene and were obtained by PCR amplification by using a primer set of poxB 5′ H3 (SEQ ID NO: 17) and DpoxB_up 3′ (SEQ ID NO: 18) and a primer set of DpoxB_dn 5′ (SEQ ID NO: 19) and poxB 3′ E1 (SEQ ID NO: 20), respectively. Products obtained from the PCR amplification were cloned between restriction enzyme sites of HindIII and EcoRI, of the vector pK19 mobsacB, thereby manufacturing a vector pK19_ΔpoxB.

The two homologous regions for removing pta-ackA gene is upstream and downstream regions of the gene and were obtained by PCR amplification by using a primer set of pta 5′ H3 (SEQ ID NO: 21) and Dpta_up_R1 3′ (SEQ ID NO: 22) and a primer set of DackA_dn_R1 5′ (SEQ ID NO: 23) and ackA 3′ Xb (SEQ ID NO: 24), respectively. Products obtained from the PCR amplification were cloned between restriction enzyme sites of HindIII and Xbal, of the vector pK19 mobsacB, thereby manufacturing a vector pK19_Δpta_ackA.

The two homologous regions for removing actA gene are upstream and downstream regions of the gene and were obtained by PCR amplification by using a primer set of actA 5′ Xb (SEQ ID NO: 25) and DactA_up_R4 3′ (SEQ ID NO: 26) and a primer set of DactA_dn_R4 5′ (SEQ ID NO: 27) and actA 3′ H3 (SEQ ID NO: 28), respectively. Products obtained from the PCR amplification were cloned between restriction enzymes, i.e., Xbal and HindIII, of the vector pK19 mobsacB, thereby manufacturing a vector pK19_ΔactA.

(2) Preparation of CGL (Δldh, ΔpoxB, Δpta-ackΔ, ΔactA)

These replacement vectors were introduced together into CGL ATCC13032 via electroporation. The introduced strains were spread over an LBHIS agar plate containing 25 μg/ml of kanamycin, and then, the plate was incubated at a temperature of 30° C. The LBHIS agar plate contains 25 g/L of Difco LB™ broth, 18.5 g/L of brain-heart infusion broth, 91 g/L of D-sorbitol, and 15 g/L of agar. Hereinafter, all the compositions of a LBHIS medium are prepared in the same manner. Colonies formed on the LBHIS medium were cultured at a temperature of 30° C. in a BHIS medium (pH 7.0) containing 37 g/L of brain heart infusion powder and 91 g/L of D-sorbitol, and the culture broth was spread over a LB/Suc10 agar plate, and then, the plate was incubated at a temperature of 3020 C. Then, colonies with double crossover were selected only. Here, the LB/Suc10 agar plate contained 25 g/L of Difco LB™ broth, 15 g/L of agar, and 100 g/L of sucrose.

Genomic DNA was isolated from the selected colonies and examined to confirm the deletion of the genes. In order to confirm the deletion of the ldh gene, a primer set of ldhA_{—}5′_HindIII and ldhA_{—}3′_EcoRI was used. In order to confirm the deletion of the poxB gene, a PCR was performed by using a primer set of poxB_up_for (SEQ ID NO: 29) and poxB_dn_rev (SEQ ID NO: 30). Additionally, in order to confirm the deletion of the pta-ackA gene, a primer set of ta_up_for (SEQ ID NO: 31) and ackA_dn_rev (SEQ ID NO: 32) was used. In order to confirm the deletion of the actA gene, a PCR was performed by using a primer set of actA_up_for (SEQ ID NO: 33) and actA_dn_rev (SEQ ID NO: 34).

(3) Preparation of CGL (Δldh, ΔpoxB, Δpta-ackA, ΔactA, pyc^{P458S})

A mutant (hereinafter referred to as ‘PYC^{P458S}’) in which proline at the 458^{th} position in the amino acid sequence of pyruvate carboxylase (PYC) (SEQ ID NO: 12) of CGL ATCC 13032 was substituted with serine was prepared.

The mutant was prepared by substituting a codon CCG that encodes proline at the 458^{th} position of the amino acid sequence of the PYC with a codon TCG, according to an overlap extension PCR method.

PCR products were obtained from genomic DNA of CGL ATCC 13032 bp using a primer set of pyc-F1 (SEQ ID NO: 35) and pyc-R1(SEQ ID NO: 36) and a primer set of pyc-F2 (SEQ ID NO: 37) and pyc-R2 (SEQ ID NO: 38). These obtained PCR products were used as templates so as to obtain another PCR product by using a primer set of pyc-F1 and pyc-R2. The finally obtained PCR product was cloned into a restriction enzyme site of Xbal, in the vector pK19mobsacB, thereby manufacturing a vector pK19mobsacB-pyc*.

The vector pK19mobsacB-pyc* was introduced into the CGL (Δldh, ΔpoxB, Δpta-ackA, ΔactA) of Example 1(2). A PCR was performed by using a primer set of pyc-F1 and pyc-R2, and sequences of the resulting PCR products were analyzed to thereby confirm the replacement of the pyc gene.

### EXAMPLE 2

### Preparation of a Gene Expression Library

(1) Preparation of a Vector pGS_Ex2

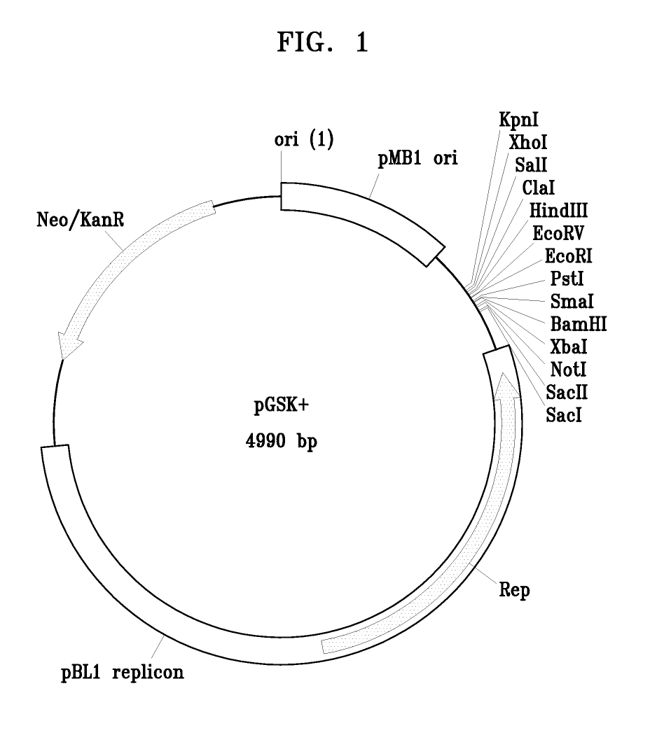

The following 4 PCR products were obtained by using Phusion High-Fidelity DNA Polymerase (New England Biolabs, cat.#M0530). PCR was performed by using a vector pET2 as a template (GenBank accession number: AJ885178.1) for screening a promoter of C. glutamicum and a primer set of MD-616 (SEQ ID NO: 39) and MD-618 (SEQ ID NO: 40) and another primer set of MD-615 (SEQ ID NO: 41) and MD-617 (SEQ ID NO: 42). In addition, another PCR was performed by using a vector pEGFP-C1 (Clontech) as a template and a primer set of MD-619 (SEQ ID NO: 43) and a MD-620 (SEQ ID NO: 44), and a vector pBluescriptII SK+ as a template and a primer set of LacZa-NR (SEQ ID NO: 45) and MD-404 (SEQ ID NO: 46). Products of each PCR, i.e., 3010 bp, 854 bp, 809 bp, and 385 bp fragments, were cloned into a circular plasmid, according to an In-Fusion EcoDry PCR Cloning Kit (Clontech, cat. #639690) method. Then, the cloned vector was transformed into a One Shot TOP10 Chemically Competent Cell (Invitrogen, cat. #C4040-06), the cells were cultured in a LB medium containing 25 mg/L of kanamycin, and growing colonies were selected. The vector was collected from the selected colonies, and the entire sequences of the vector were identified by analyzing the entire sequence of the vector. The vector was assigned as pGSK+ (see FIG. 1).

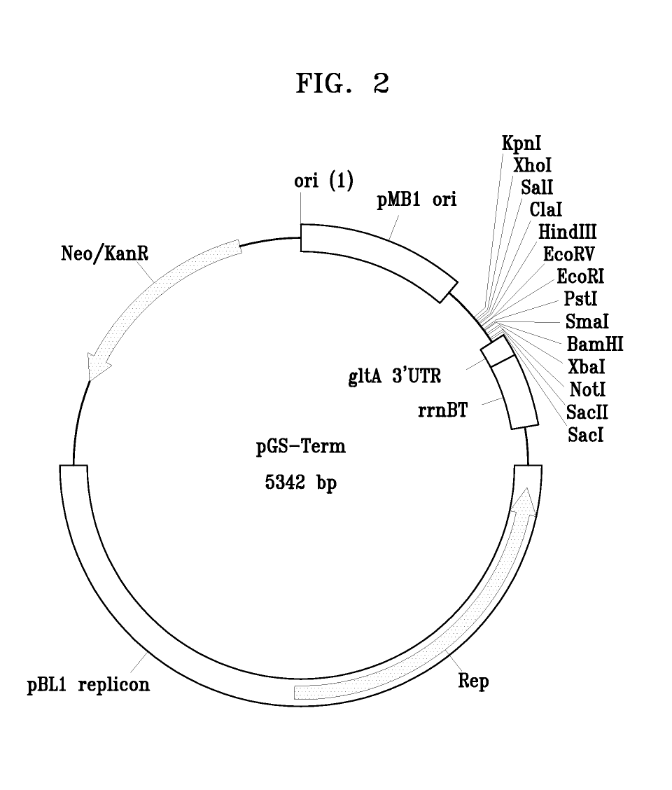

In addition, 3′UTR of C. glutamicum gltA (NCgl0795) and rho-independent terminator of E. coli rrnB were inserted into the vector pGSK+as follows. A 108 bp PCR fragment of the gltA 3′UTR was obtained by performing a PCR by using the genomic DNA of C. glutamicum ATCC13032 as a template and a primer set of MD-627 (SEQ ID NO: 47) and MD-628 (SEQ ID NO: 48). Furthermore, a 292 bp PCR fragment of rrnB translation terminator was obtained by using the genomic DNA of E. coli (MG1655) as a template and a primer set of MD-629 (SEQ ID NO: 49) and MD-630 (SEQ ID NO: 50). These two fragments were inserted into the vector pGSK+ cleaved by Sad by using an In-Fusion EcoDry PCR Cloning Kit (Clontech, cat. #639690). The cloned vector was then transformed into a One Shot TOP10 Chemically Competent Cell (Invitrogen, cat. #C4040-06), the cells were cultured in a LB medium containing 25 mg/L of kanamycin, and growing colonies were selected. The vector was collected from the selected colonies, and sequences of the vector were identified by analyzing the entire sequence of the vector. The vector was assigned as pGS-Term (see FIG. 2).

211 bp DNA fragment was obtained by using the genomic DNA of C. glutamicum ATCC13032 as a template and a primer set of MD-1452 (SEQ ID NO: 51) and MD-1453 (SEQ ID NO: 52) via a PCR by using Phusion High-Fidelity DNA Polymerase (New England Biolabs, cat. #M0530). The DNA fragment was mixed with the vector pGS-Term cleaved by a restriction enzyme, KpnI and cloned into a circular plasmid, according to an In-Fusion EcoDry PCR Cloning Kit (Clonetech, cat. #639690) method. Then, the cloned vector was transformed into a One Shot TOP10 Chemically Competent Cell (Invitrogen, cat. #C4040-06), the cells were cultured in a LB medium containing 25 mg/L of kanamycin, and growing colonies were selected. The vector was collected from the selected colonies, and sequences of the vector were identified by analyzing the entire sequence of the vector. The vector was assigned as pGS-Ex2.

The vector pGS-Ex2 includes a promoter of NCgl1929 gene and an artificial ribosome binding site (RBS), as well as 3′UTR of C. glutamicum gltA and a terminator of E. coli rrnB. Additionally, a multi-cloning site (MCS) is present between the promoter-RBS and the 3′UTR-terminator.

(2) Preparation of a Vector and a Library for Gene Expression

(1) A total 64 types of genes including carbon metabolic genes of C. glutamicum were each cloned into a site between KpnI and SacI in the vector pGS-EX2 of (1). Gene expression vectors corresponding to the total 64 types of genes were prepared by locating C-terminal His₆-tag sequence for confirmation of protein expression behind the ORF thereof.

The total 64 types of genes used herein are shown in Table 1 below. Here, ID* indicates entry number of KEGG (Kyoto Encyclopedia of Genes and Genomes) database. The expression Ec. or Kp. ahead of the name of the gene represents that the gene is derived from E. coli or Klebsiella pneumoniae, respectively. The EGFP gene disclosed in research paper of Cormack et al. in 1996 (Cormack B P, Valdivia R H, Falkow S (1996) “FACS-optimized mutants of the green fluorescent protein (GFP)” Gene 173(1): 33-38) was cloned from a vector pEGFP-C1 from Clontech (cat. #6048-1). The tagRFP gene was cloned from a vector pTagRFP-C from Evrogen (cat. #FP141). The sucA gene was derived from Mycobacterium bovis, and the Kp.lpd gene was derived from K pneumoniae.

In a vector pGS-Ex2 EGFP-H6 used as a control, the EGFP gene was obtained by PCR amplification by using a vector MD0545 as a template, which is a C-terminal His×6 epitope tagging vector manufactured by the following method, and a primer set of MD-1557 (SEQ ID NO: 53) and MD-1620(SEQ ID NO: 54). A 753 bp DNA fragment (SEQ ID NO: 57) was obtained by PCR amplification using a vector pEGFP-C1 from Clonetech (cat #6048-1) as a template and a primer set of MD-1357 (SEQ ID NO: 55) and MD-1358 (SEQ ID NO: 56). The DNA fragment was ligated into a fragment of a vector pET28a from Novagen (cat. #69864-3) cleaved by BamHI and XhoI by using an In-fusion EcoDry PCR Cloning Kit from Clontech (cat. #639690), thereby manufacturing a vector MD0545 (SEQ ID NO: 58). The EGFP gene obtained therefrom was ligated into a fragment of the vector pGS-Ex2 cleaved by KpnI and SacI by using an In-fusion EcoDry PCR Cloning Kit from Clontech (cat. #639690), thereby manufacturing a control group vector pGS-Ex2 EGFP-H6.

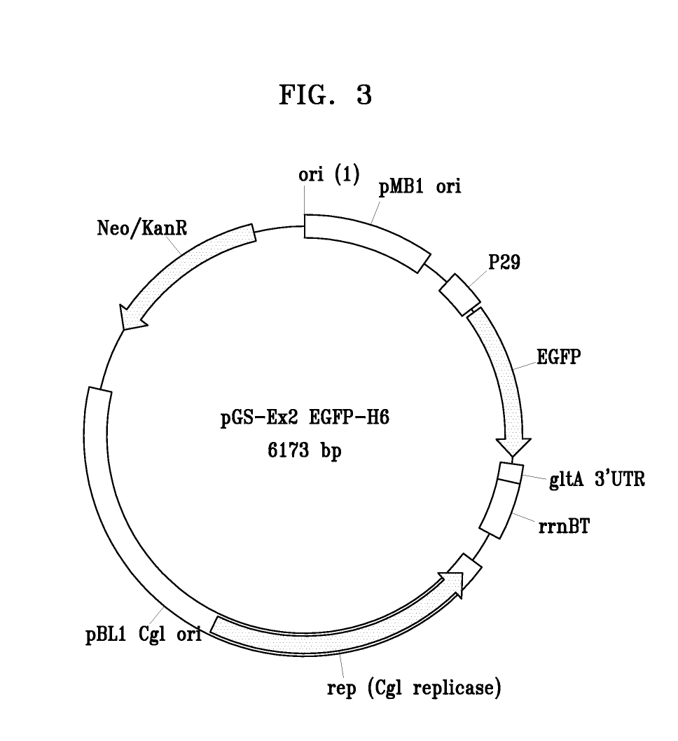

FIG. 3 is a map of a vector pGS-Ex2 EGFP-H6.

TABLE 1

| Gene | ID* or vector | Gene | ID* or vector |
|---|---|---|---|
| aceA | NCgl2248 | lpdA | NCgl0658 |
| aceB | NCgl2247 | mdh | NCgl2297 |
| aceE | NCgl2167 | mdh2 | NCgl0631 |
| aceF | NCgl2126 | mez | NCgl2904 |
| ackA | NCgl2656 | mqo | NCgl1926 |
| acn | NCgl1482 | odhA | NCgl1084 |
| actA | NCgl2480 | odx | NCgl1241 |
| devB | NCgl1516 | pckG | NCgl2765 |
| Ec.aceE | b0114 | pfkA | NCgl1202 |
| Ec.aceF | b0115 | pgi | NCgl0817 |
| Ec.lpd | b0116 | pgk | NCgl1525 |
| Ec.lpd(E354K) | b0116** | poxB | NCgl2521 |
| EGFP | pEGFP-C1 | ppc | NCgl1523 |
| eno | NCgl0935 | pps | NCgl0529 |
| fda | NCgl2673 | prpC1 | NCgl0666 |
| fumC | NCgl0967 | pta | NCgl2657 |
| gabD1 | NCgl2619 | pyc | NCgl0659 |
| gabD2 | NCgl0463 | pyk | NCgl2008 |
| gabD3 | NCgl0049 | rpe | NCgl1536 |
| gapA | NCgl1526 | rpiA | NCgl2337 |
| gapB | NCgl0900 | sdhA | NCgl0360 |
| gdh | NCgl1999 | sdhB | NCgl0361 |
| glnA | NCgl2133 | sdhCD | NCgl0359 |
| gInA2 | NCgl2148 | sucA | NP_854934 |
| gltA | NCgl0795 | sucC | NCgl2477 |
| gltB | NCgl0181 | sucD | NCgl2476 |
| gnd | NCgl1396 | sucE | NCgl0213 |
| gpmA | NCgl0390 | tagRFP | pTagRFP-C |
| icd | NCgl0634 | tal | NCgl1513 |
| Kp.lpd(E354K) | KPN_00120** | tkt | NCgl1512 |
| ldhA | NCgl2810 | tpiA | NCgl1524 |
| lpd | NCgl0355 | zwf | NCgl1514 |

**indicates entry number of KEGG regarding its wild-type.

The vector was introduced into CGL strain (Δldh, ΔpoxB, Δpta-ackA, ΔactA, pyc^{P458S}) of Example 1 according to research paper of van der Rest et al. (van der Rest M E, Lange C, Molenaar D (1999) “A heat shock following electroporation induces highly efficient transformation of Corynebacterium glutamicum with xenogeneic plasmid DNA” Appl Microbiol Biotechnol. 52(4): 541-545).

That is, 10 mL of a culture broth cultured overnight in LBG medium (10 g/L of tripton, 5 g/L of yeast extract, 10 g/L of NaCl, 20 g/L of glucose) was poured into 100 mL of Epo medium (10 g/L of tripton, 5 g/L of yeast extract, 10 g/L of NaCl, 4 g/L of isonicotinic acid hydrazide, 25 g/L of glycine, 0.1% of Tween 80), and then, cultured at a temperature of 18° C. at a speed of 120 rpm for 28 hours. Grown cells were cooled with ice for 10 minutes, washed out 4 times with 10% glycerol solution at 0° C., and finally suspended in 500 μl of 10% glycerol solution. 1 μg of vector DNA was mixed with 100 μl of the cell suspension and subjected to electroporation at 25 μl and 2.5 kV. Afterwards, 1 mL of BHIS medium (37 g/L of brain-heart infusion broth, 91 g/L of D-sorbitol, pH 7.0) at 46° C. was added thereto and the mixture was cultured for 6 minutes at 46° C. Hereinafter, all the compositions of BHIS medium are prepared in the same manner. The resulting broth was again stirred and cultured at 30° C. and 10 μl of the culture broth was spread over LBHIS/Kan agar plate (5 g/L of tripton, 2.5 g/L of yeast extract, 5 g/L of NaCl, 18.5 g/L of brain-heart infusion broth, 91 g/L of D-sorbitol, 15 g/L of agar, 25 mg/L of kanamycin) and cultured at 30° C. for 48 hours. Hereinafter, all the compositions of a LBHIS/Kan agar medium are prepared in the same manner.

As a result, expression library of the 64 types of genes was prepared.

### EXAMPLE 3

### Experiments Regarding Culturing Conditions

Each strain of the gene expression library prepared in Example 2 was inoculated into 5 mL of BHIS medium containing 25 μg/ml of kanamycin, and was stirred and cultured at 3020 C. for 16 hours. Afterwards, under various culturing conditions (4HB, m4HB, Saline, CB, CB+CC, CGXII, and CGXII/Ooxy), yields of succinate were compared therebetween.

Colonies of C. glutamicum formed on the LBHIS/Kan agar medium were inoculated into 3 mL of LB broth containing 25 mg/L of kanamycin, and the broth was stirred overnight at 3020 C., thereby performing a seed culture.

Regarding 4HB, the seed culture broth was centrifuged at room temperature at 3,700 rpm for 5 minutes to obtain cells. The obtained cells were re-suspended in 25 mL of a fermentation medium having the composition of 4HB and m4HB in Table 2, and then, cultured in a 125 mL vented cap flask at 3020 C. for 24 hours. Afterwards, the cell culture was transferred to a glass vial and the glass vial was sealed. Anaerobic culture was carried out thereto for 24 hours again. After completion of culture, cells were obtained therefrom, and the supernatant was analyzed by HPLC.

Regarding m4HB, Saline, CB, CB+CC, CGXII, and CGXII/0oxy, the seed culture broth was inoculated into 30 mL of LB broth containing 25 mg/L of kanamycin, and then, cultured for 5 hours until OD₆₀₀ becomes greater than 3.0. Cells obtained therefrom were washed out 1 time with each medium having the composition listed in Table 2, and then, re-suspended in 1 mL of each medium. After each suspension was diluted until OD₆₀₀ of final 1 mL broth reached 30.0 and then transferred to a 12-well microplate. Afterwards, in the case of m4HB, Saline, CB, CB+CC, and CGXII under 2.5% oxygen conditions, or in the case of CGXII/0oxy under 100% nitrogen conditions, culturing was performed under stirring for 24 hours. After completion of culturing, cells were obtained therefrom, and supernatant was analyzed by HPLC.

TABLE 2

| Culturing | | |
|---|---|---|
| conditions | Medium compositions | Remarks |
| 4HB and | 40 g/L D-glucose, 10 g/L corn steep liquor | |
| m4HB | (CSL), 2 g/L (NH₄)₂SO₄, 1 g/L KH₂PO₄, | |
| | 0.5 g/L MgSO₄-7H₂O, 10 mg/L | |
| | FeSO₄-7H₂O, 10 mg/L MnSO₄-H₂O, | |
| | 0.1 mg/L ZnSO₄-7H₂O, 0.1 mg/L | |
| | CuSO₄-5H₂O, 3 mg/L thiamine-HCl, | |
| | 0.3 mg/L biotin, 1 mg/L Ca-pantothenate, | |
| | 5 mg/L nicotinamide, 30 g/L CaCO₃, | |
| | pH 7.0 | |
| Saline | 0.9% NaCl, 4% D-glucose | |
| CB | 42 g/L 3-morpholinepropanesulfonic | |
| | acid (MOPS), 1 g/L KH₂PO₄, 1 g/L | |
| | K₂HPO₄, 4% D-glucose | |
| CB + CC | 42 g/L MOPS, 1 g/L KH₂PO₄, 1 g/L | |
| | K₂HPO₄, 4% D-glucose, 1% CaCO₃ | |
| CGXII and | 20 g/L (NH₄)₂SO₄, 5 g/L urea, 1 g/L | Litsanov, et |
| CGXII/Doxy | KH₂PO₄, 1 g/L K₂HPO₄, 0.25 g/L | al., 2012 |
| | MgSO₄•H₂O, 10 mg/L CaCl₂, 10 mg/L | |
| | FeSO₄•H₂O, 0.1 mg/L MnSO₄•H₂O, | |
| | 1 mg/L ZnSO₄•H₂O, 0.2 mg/L | |
| | CuSO₄•H₂O, 20 mg/L NiCl₂•H₂O, | |
| | 0.2 mg/L biotin, 42 g/L MOPS, | |
| | 4%(w/v) glucose | |

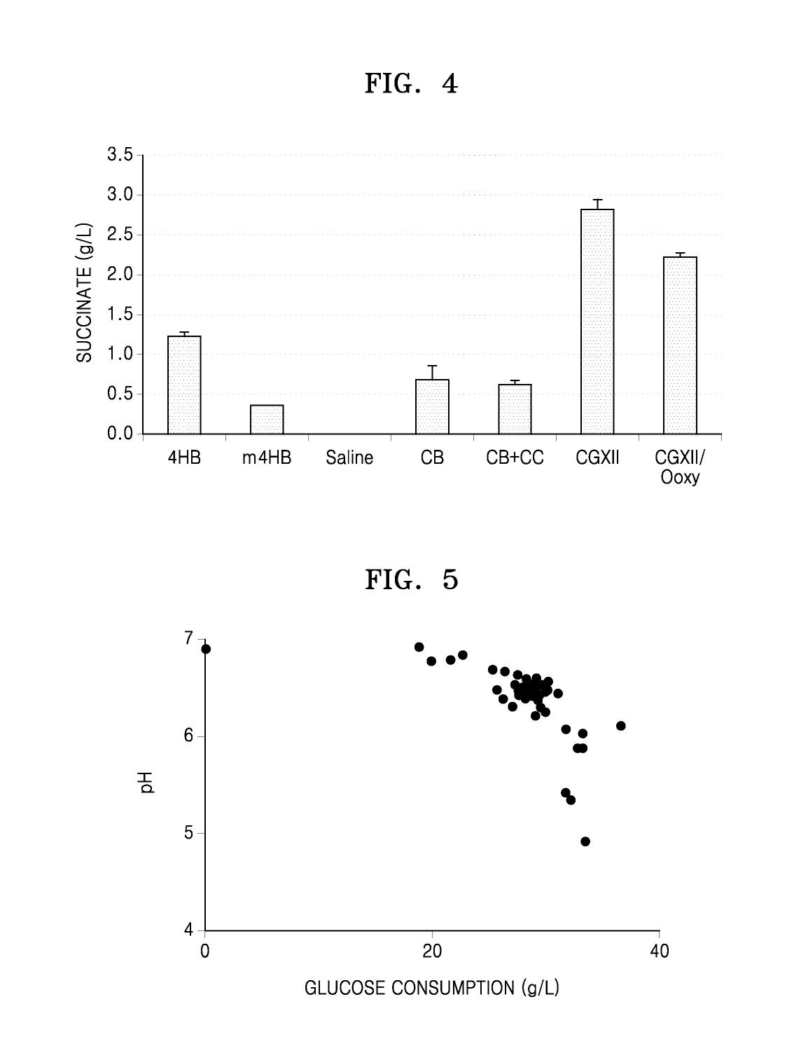

FIG. 4 is a graph showing yields of succinate under culturing conditions of 4HB, m4HB, Saline, CB, CB+CC, CGXII, and CGXII/0oxy. As a result, it was found that the yield of succinate was the highest in a CGXII culturing condition.

### EXAMPLE 4

### Evaluation of Succinate Productivity 1

On the basis of experimental results of culturing conditions of Example 3, the gene expression library of Example 2 was cultured in a CGXII culturing condition.

30 or more colonies were collected for each strain and inoculated into 5 mL of BHIS medium containing 25 μg/ml of kanamycin, and then, cultured at 3020 C. under stirring for 16 hours. Afterwards, the culture was inoculated into 20 mL of BHIS medium containing 25 μg/ml of kanamycin, and then, cultured at 3020 C. under stirring for 6 hours. The cultured cells were collected and washed out with a CGXII medium at least 1 time, and then, diluted until OD₆₀₀ reached 30.0 in the CGXII medium. The cell suspension was cultured in an incubator, in which atmospheric oxygen concentration is maintained at a level of 2.5%, at 3020 C. under stirring for 20 hours. After completion of the culture, the supernatant in which all cells were removed by centrifugation and filtration were collected and subjected to HPLC analysis, thereby measuring yields of succinate and residual glucose amounts.

Additionally, the collected supernatant was subjected to a reaction with chlorophenol red, and then, optical density (OD) thereof was analyzed at a wavelength between 435 nm and 575 nm, thereby measuring a final pH of the supernatant. FIG. 5 is a graph showing a correlation between glucose consumption and ultimate pH. As shown in FIG. 5, the pH of the supernatant was found to be influenced by glucose consumption, and from this, the present experimental results is reliable.

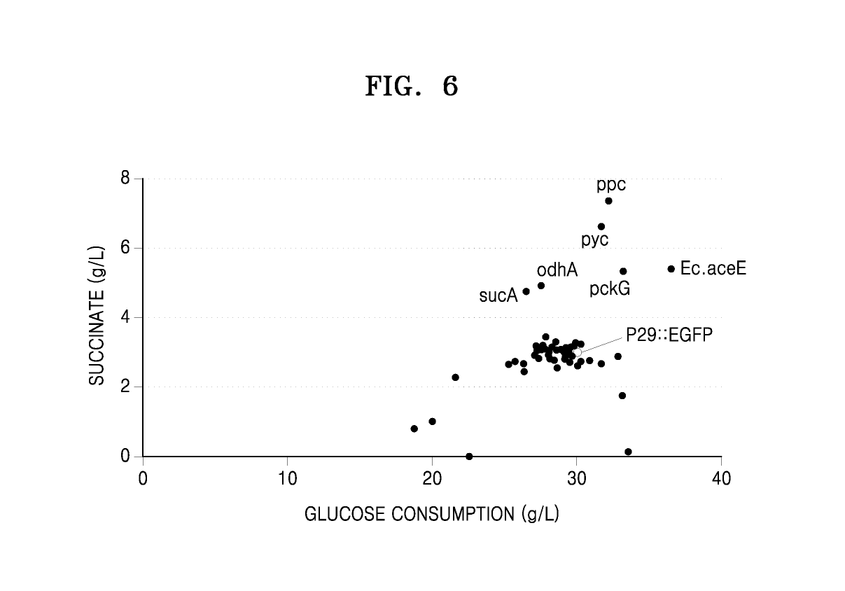

FIG. 6 is a graph showing screening results of succinate productivity and indicating major genes. As a result, it was confirmed that, for CGL strains which overexpress ppc, pyc, Ec.aceE, pckG, odhA or sucA gene, the yields of succinate was better than that of a control EGFP strain.

### EXAMPLE 5

### Evaluation of Succinate Productivity 2

In a CGXII culturing condition of Example 4, for the strains whose yield of succinate was increased by 50% or higher compared to that of a control, the productivity of succinate was evaluated in a fermentation medium. The medium compositions used herein are the same with those in 4HB and m4HB culturing condition shown in Table 2. Culturing conditions other than the medium compositions were the same with those performed in Example 3. After completion of the culturing, the supernatant in which all cells were removed by centrifugation and filtration were collected and subjected to HPLC analysis, thereby measuring yields of succinate and residual glucose amounts.

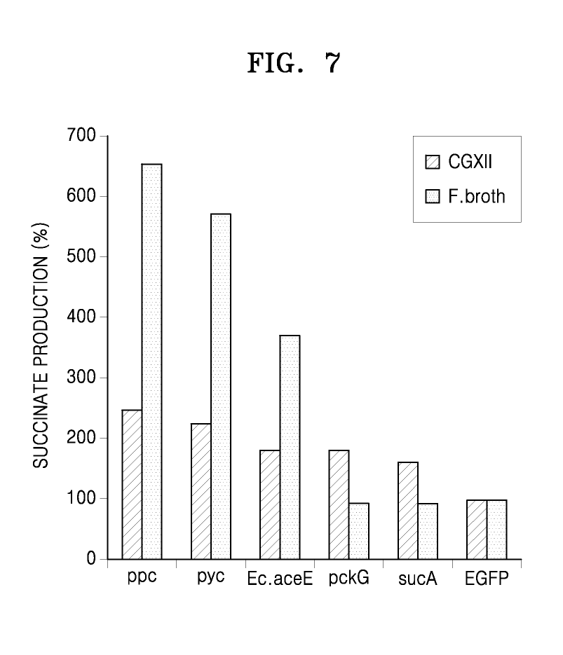

FIG. 7 is a graph showing succinate productivity in a CGXII medium and a fermentation medium. For Ec.aceE, the amount of glucose consumption in the CGXII medium was increased by 21.7% and the yield of succinate was increased by 84.8%, compared with a control strain, EGFP. In addition, the amount of glucose consumption in the fermentation medium was increased by 2.8% and the yield of succinate was increased by 372.9%, compared with a control strain, EGFP. Thus, it was confirmed that the overexpression of Ec.aceE gene in C. glutamicum significantly improved succinate productivity.

As described above, according to the one or more of the above embodiments of the present invention, a microorganism in which an exogenous pyruvate dehydrogenase E1 is contained and/or expression of an alpha-ketoglutarate dehydrogenase E1 is increased may be used to efficiently produce succinate according a method of producing succinate.

It should be understood that the exemplary embodiments described therein should be considered in a descriptive sense only and not for purposes of limitation. Descriptions of features or aspects within each embodiment should typically be considered as available for other similar features or aspects in other embodiments.

While one or more embodiments of the present invention have been described with reference to the figures, it will be understood by those of ordinary skill in the art that various changes in form and details may be made therein without departing from the spirit and scope of the present invention as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant microorganism comprising
(a) an exogenous pyruvate dehydrogenase E1;
(b) increased expression of alpha-ketoglutarate dehydrogenase E1 compared to a reference microorganism,
or both (a) and (b).

2. The recombinant microorganism of claim 1, wherein the microorganism belongs to the genus Corynebacterium.

3. The recombinant microorganism of claim 1, wherein the exogenous pyruvate dehydrogenase E1 is from Escherichia coli.

4. The recombinant microorganism of claim 1, wherein the exogenous pyruvate dehydrogenase E1 comprises SEQ ID NO: 1.

5. The recombinant microorganism of claim 1, wherein the increased expression of the alpha-ketoglutarate dehydrogenase E1 compared to a reference microorganism is caused by increased expression of an endogenous polynucleotide that encodes the alpha-ketoglutarate dehydrogenase E1 in the recombinant microorganism.

6. The recombinant microorganism of claim 1, wherein the increased expression of the alpha-ketoglutarate dehydrogenase E1 component compared to a reference microorganism is caused by the introduction of a polynucleotide that encodes the alpha-ketoglutarate dehydrogenase E1 component into the recombinant microorganism.

7. The recombinant microorganism of claim 1, wherein the alpha-ketoglutarate dehydrogenase E1 comprises SEQ ID NO: 2.

8. The recombinant microorganism of claim 7, wherein the alpha-ketoglutarate dehydrogenase E1 is encoded by the polynucleotide comprising SEQ ID NO: 3.

9. The recombinant microorganism of claim 1, wherein expression of phosphoenolpyruvate carboxylase, phosphoenolpyruvate carboxykinase, alpha-ketoglutarate decarboxylase, or a combination thereof is increased in the recombinant microorganism as compared to a reference microorganism.

10. The recombinant microorganism of claim 1, wherein an activity of converting pyruvate into lactate, an activity of converting acetyl-coA into acetate, or a combination thereof is removed or reduced in the recombinant microorganism compared to a reference microorganism.

11. The recombinant microorganism of claim 10, wherein a polynucleotide encoding L-lactate dehydrogenase, a polynucleotide encoding pyruvate oxidase, a polynucleotide encoding phosphotransacetylase, a polynucleotide encoding acetate kinase, a polynucleotide encoding acetate coenzyme A-transferase, or a combination thereof is inactivated or attenuated in the recombinant microorganism compared to a reference microorganism.

12. The recombinant microorganism of claim 12, wherein the polynucleotide encoding L-lactate dehydrogenase, the polynucleotide encoding pyruvate oxidase, the polynucleotide encoding phosphotransacetylase, the polynucleotide encoding acetate kinase, and the polynucleotide encoding acetate coenzyme A-transerase encode amino acid sequences of SEQ ID NOs: 7 to 11, respectively.

13. The recombinant microorganism of claim 1, wherein an activity of catalyzing the conversion of pyruvate into oxaloacetate is increased in the recombinant microorganism as compared to a reference microorganism.

14. The recombinant microorganism of claim 13, wherein the pyruvate carboxylase comprises SEQ ID NO: 12 modified by a P458S substitution.

15. A method of producing succinate, the method comprising:
culturing the recombinant microorganism of claim 1 in a cell culture medium, whereby the recombinant microorganism produces succinate; and
collecting succinate from the cultured microorganism.

16. The method of claim 15, wherein the culturing is performed under anaerobic conditions.

17. A method of increasing succinate production in a micoorgansim by
(a) introducing an exogenous polynucleotide encoding pyruvate dehydrogenase E1 into the microorganism;
(b) increasing the expression of alpha-ketoglutarate dehydrogenase E1 in the microorganism,
or both (a) and (b).

18. The method of claim 17, wherein the microorganism belongs to the genus Corynebacterium.

19. The method of claim 17, wherein the exogenous polynucleotide encoding pyruvate dehydrogenase E1 is from Escherichia coli.

20. The method of claim 1, wherein the pyruvate dehydrogenase E1 comprises SEQ ID NO: 1, and the alpha-ketoglutarate dehydrogenase E1 comprises SEQ ID NO: 2.


SEQUENCE LISTING (58 sequences)

SEQ ID NO: 1 - protein, 887 aa, Escherichia coli
MSERFPNDVDPIETRDWLQAIESVIREEGVERAQYLIDQLLAEARKGGVNVAAGTGISNYINTIPVEEQPEYPGNLELERRIRSAIRWNAIMTVLRASKKDLELGGHMASFQSSATIYDVCFNHFFRARNEQDGGDLVYFQGHISPGVYARAFLEGRLTQEQLDNFRQEVHGNGLSSYPHPKLMPEFWQFPTVSMGLGPIGAIYQAKFLKYLEHRGLKDTSKQTVYAFLGDGEMDEPESKGAITIATREKLDNLVFVINCNLQRLDGPVTGNGKIINELEGIFEGAGWNVIKVMWGSRWDELLRKDTSGKLIQLMNETVDGDYQTFKSKDGAYVREHFFGKYPETAALVADWTDEQIWALNRGGHDPKKIYAAFKKAQETKGKATVILAHTIKGYGMGDAAEGKNIAHQVKKMNMDGVRHIRDRFNVPVSDADIEKLPYITFPEGSEEHTYLHAQRQKLHGYLPSRQPNFTEKLELPSLQDFGALLEEQSKEISTTIAFVRALNVMLKNKSIKDRLVPIIADEARTFGMEGLFRQIGIYSPNGQQYTPQDREQVAYYKEDEKGQILQEGINELGAGCSWLAAATSYSTNNLPMIPFYIYYSMFGFQRIGDLCWAAGDQQARGFLIGGTSGRTTLNGEGLQHEDGHSHIQSLTIPNCISYDPAYAYEVAVIMHDGLERMYGEKQENVYYYITTLNENYHMPAMPEGAEEGIRKGIYKLETIEGSKGKVQLLGSGSILRHVREAAEILAKDYGVGSDVYSVTSFTELARDGQDCERWNMLHPLETPRVPYIAQVMNDAPAVASTDYMKLFAEQVRTYVPADDYRVLGTDGFGRSDSRENLRHHFEVDASYVVVAALGELAKRGEIDKKVVADAIAKFNIDADKVNPRLA

SEQ ID NO: 2 - protein, 1221 aa, Corynebacterium glutamicum
MSSASTFGQNAWLVDEMFQQFQKDPKSVDKEWRELFEAQGGPNTTPATTEAQPSAPKESAKPAPKAAPAAKAAPRVETKPADKTAPKAKESSVPQQPKLPEPGQTPIRGIFKSIAKNMDISLEIPTATSVRDMPARLMFENRAMVNDQLKRTRGGKISFTHIIGYAMVKAVMAHPDMNNSYDVIDGKPTLIVPEHINLGLAIDLPQKDGSRALVVAAIKETEKMNFSEFLAAYEDIVARSRKGKLTMDDYQGVTVSLTNPGGIGTRHSVPRLTKGQGTIIGVGSMDYPAEFQGASEDRLAELGVGKLVTITSTYDHRVIQGAVSGEFLRTMSRLLTDDSFWDEIFDAMNVPYTPMRWAQDVPNTGVDKNTRVMQLIEAYRSRGHLIADTNPLSWVQPGMPVPDHRDLDIETHNLTIWDLDRTFNVGGFGGKETMTLREVLSRLRAAYTLKVGSEYTHILDRDERTWLQDRLEAGMPKPTQAEQKYILQKLNAAEAFENFLQTKYVGQKRFSLEGAEALIPLMDSAIDTAAGQGLDEVVIGMPHRGRLNVLFNIVGKPLASIFNEFEGQMEQGQIGGSGDVKYHLGSEGQHLQMFGDGEIKVSLTANPSHLEAVNPVMEGIVRAKQDYLDKGVDGKTVVPLLLHGDAAFAGLGIVPETINLAKLRGYDVGGTIHIVVNNQIGFTTTPDSSRSMHYATDYAKAFGCPVFHVNGDDPEAVVWVGQLATEYRRRFGKDVFIDLVCYRLRGHNEADDPSMTQPKMYELITGRETVRAQYTEDLLGRGDLSNEDAEAVVRDFHDQMESVFNEVKEGGKKQAEAQTGITGSQKLPHGLETNISREELLELGQAFANTPEGFNYHPRVAPVAKKRVSSVTEGGIDWAWGELLAFGSLANSGRLVRLAGEDSRRGTFTQRHAVAIDPATAEEFNPLHELAQSKGNNGKFLVYNSALTEYAGMGFEYGYSVGNEDSIVAWEAQFGDFANGAQTIIDEYVSSGEAKWGQTSKLILLLPHGYEGQGPDHSSARIERFLQLCAEGSMTVAQPSTPANHFHLLRRHALSDLKRPLVIFTPKSMLRNKAAASAPEDFTEVTKFQSVINDPNVADAAKVKKVMLVSGKLYYELAKRKEKDGRDDIAIVRIEMLHPIPFNRISEALAGYPNAEEVLFVQDEPANQGPWPFYQEHLPELIPNMPKMRRVSRRAQSSTATGVAKVHQLEEKQLIDEAFEA

SEQ ID NO: 3 - DNA, 3666 bp, Corynebacterium glutamicum
gtgagcagcgctagtactttcggccagaatgcgtggctggtagacgagatgttccagcagttccagaaggaccccaagtccgtggacaaggaatggagagaactctttgaggcgcaggggggaccaaatactacccccgctacaacagaagcacagccttcagcgcccaaggagtctgcgaaaccagcaccaaaggctgcccctgcagccaaggcagcaccgcgcgtagaaaccaagccggccgacaagaccgcccctaaggccaaggagtcctcagtgccacagcaacctaagcttccggagccaggacaaaccccaatcaggggtattttcaagtccatcgcgaagaacatggatatctccctggaaatcccaaccgcaacctcggttcgcgatatgccagctcgcctcatgttcgaaaaccgcgcgatggtcaacgatcagctcaagcgcacccgcggtggcaagatctccttcacccacatcattggctacgccatggtgaaggcagtcatggctcacccggacatgaacaactcctacgacgtcatcgacggcaagccaaccctgatcgtgcctgagcacatcaacctgggccttgctatcgaccttcctcagaaggacggctcccgcgcacttgtcgtagcagccatcaaggaaaccgagaagatgaacttctccgagttcctcgcagcctacgaagacatcgtggcacgctcccgcaagggcaagctcaccatggatgactaccagggcgttaccgtttccttgaccaacccaggtggcatcggtacccgccactctgttccacgtctaaccaagggccagggcaccatcatcggtgtcggttccatggattacccagcagagttccagggcgcttcagaagaccgccttgcagagctcggcgttggcaaacttgtcaccatcacctccacctacgatcaccgcgtgatccagggtgctgtgtccggtgaattcctgcgcaccatgtctcgcctgctcaccgatgattccttctgggatgagatcttcgacgcaatgaacgttccttacaccccaatgcgttgggcacaggacgttccaaacaccggtgttgataagaacacccgcgtcatgcagctcattgaggcataccgctcccgtggacacctcatcgctgacaccaacccactttcatgggttcagcctggcatgccagttccagaccaccgcgacctcgacatcgagacccacaacctgaccatctgggatctggaccgtaccttcaacgtcggtggcttcggcggcaaggagaccatgaccctgcgcgaggtactgtcccgcctccgcgctgcgtacaccctcaaggtcggctccgaatacacccacatcctggaccgcgacgagcgcacctggctgcaggaccgcctcgaggccggaatgccaaagccaacccaggcagagcagaagtacatcctgcagaagctgaacgccgcggaggctttcgagaacttcctgcagaccaagtacgtcggccagaagcgcttctccctcgaaggtgcagaagcacttatcccactgatggactccgccatcgacaccgccgcaggccaaggcctcgacgaagttgtcatcggtatgccacaccgtggtcgcctcaacgtgctgttcaacatcgtgggcaagccactggcatccatcttcaacgagtttgaaggccaaatggagcagggccagatcggtggctccggtgacgtgaagtaccacctcggttccgaaggccagcacctgcagatgttcggcgacggcgagatcaaggtctccctgactgctaacccgtcccacctggaagctgttaacccagtgatggaaggtatcgtccgcgcaaagcaggactacctggacaagggcgtagacggcaagactgttgtgccactgctgctccacggtgacgctgcattcgcaggcctgggcatcgtgccagaaaccatcaacctggctaagctgcgtggctacgacgtcggcggcaccatccacatcgtggtgaacaaccagatcggcttcaccaccaccccagactccagccgctccatgcactacgcaaccgactacgccaaggcattcggctgcccagtcttccacgtcaacggcgacgacccagaggcagttgtctgggttggccagctggccaccgagtaccgtcgtcgcttcggcaaggacgtcttcatcgacctcgtctgctaccgcctccgcggccacaacgaagctgatgatccttccatgacccagccaaagatgtatgagctcatcaccggccgcgagaccgttcgtgctcagtacaccgaagacctgctcggacgtggagacctctccaacgaagatgcagaagcagtcgtccgcgacttccacgaccagatggaatctgtgttcaacgaagtcaaggaaggcggcaagaagcaggctgaggcacagaccggcatcaccggctcccagaagcttccacacggccttgagaccaacatctcccgtgaagagctcctggaactgggacaggctttcgccaacaccccagaaggcttcaactaccacccacgtgtggctcccgttgctaagaagcgcgtctcctctgtcaccgaaggtggcatcgactgggcatggggcgagctcctcgccttcggttccctggctaactccggccgcttggttcgccttgcaggtgaagattcccgccgcggtaccttcacccagcgccacgcagttgccatcgacccagcgaccgctgaagagttcaacccactccacgagcttgcacagtccaagggcaacaacggtaagttcctggtctacaactccgcactgaccgagtacgcaggcatgggcttcgagtacggctactccgtaggaaacgaagactccatcgttgcatgggaagcacagttcggcgacttcgccaacggcgctcagaccatcatcgatgagtacgtctcctcaggcgaagctaagtggggccagacctccaagctgatccttctgctgcctcacggctacgaaggccagggcccagaccactcttccgcacgtatcgagcgcttcctgcagctgtgcgctgagggttccatgactgttgctcagccatccaccccagcaaaccacttccacctactgcgtcgtcacgctctgtccgacctgaagcgtccactggttatcttcaccccgaagtccatgctgcgtaacaaggctgctgcctccgcaccagaagacttcactgaggtcaccaagttccagtccgtgatcaacgatccaaacgttgcagatgcagccaaggtgaagaaggtcatgctggtctccggcaagctgtactacgaattggcaaagcgcaaggagaaggacggacgcgacgacatcgcgatcgttcgtatcgaaatgctccacccaattccgttcaaccgcatctccgaggctcttgccggctaccctaacgctgaggaagtcctcttcgttcaggatgagccagcaaaccagggcccatggccgttctaccaggagcacctcccagagctgatcccgaacatgccaaagatgcgccgcgtttcccgccgcgctcagtcctccaccgcaactggtgttgccaaggtgcaccagctggaggagaagcagcttatcgacgaggctttcgaggcttaa

SEQ ID NO: 4 - protein, 919 aa, Corynebacterium glutamicum
MTDFLRDDIRFLGQILGEVIAEQEGQEVYELVEQARLTSFDIAKGNAEMDSLVQVFDGITPAKATPIARAFSHFALLANLAEDLYDEELREQALDAGDTPPDSTLDATWLKLNEGNVGAEAVADVLRNAEVAPVLTAHPTETRRRTVFDAQKWITTHMRERHALQSAEPTARTQSKLDEIEKNIRRRITILWQTALIRVARPRIEDEIEVGLRYYKLSLLEEIPRINRDVAVELRERFGEGVPLKPVVKPGSWIGGDHDGNPYVTAETVEYSTHRAAETVLKYYARQLHSLEHELSLSDRMNKVTPQLLALADAGHNDVPSRVDEPYRRAVHGVRGRILATTAELIGEDAVEGVWFKVFTPYASPEEFLNDALTIDHSLRESKDVLIADDRLSVLISAIESFGFNLYALDLRQNSESYEDVLTELFERAQVTANYRELSEAEKLEVLLKELRSPRPLIPHGSDEYSEVTDRELGIFRTASEAVKKFGPRMVPHCIISMASSVTDVLEPMVLLKEFGLIAANGDNPRGTVDVIPLFETIEDLQAGAGILDELWKIDLYRNYLLQRDNVQEVMLGYSDSNKDGGYFSANWALYDAELQLVELCRSAGVKLRLFHGRGGTVGRGGGPSYDAILAQPRGAVQGSVRITEQGEIISAKYGNPETARRNLEALVSATLEASLLDVSELTDHQRAYDIMSEISELSLKKYASLVHEDQGFIDYFTQSTPLQEIGSLNIGSRPSSRKQTSSVEDLRAIPWVLSWSQSRVMLPGWFGVGTALEQWIGEGEQATQRIAELQTLNESWPFFTSVLDNMAQVMSKAELRLAKLYADLIPDTEVAERVYSVIREEYFLTKKMFCVITGSDDLLDDNPLLARSVQRRYPYLLPLNVIQVEMMRRYRKGDQSEQVSRNIQLTMNGLSTALRNSG

SEQ ID NO: 5 - protein, 610 aa, Corynebacterium glutamicum
MTTAAIRGLQGEAPTKNKELLNWIADAVELFQPEAVVFVDGSQAEWDRMAEDLVEAGTLIKLNEEKRPNSYLARSNPSDVARVESRTFICSEKEEDAGPTNNWAPPQAMKDEMSKHYAGSMKGRTMYVVPFCMGPISDPDPKLGVQLTDSEYVVMSMRIMTRMGIEALDKIGANGSFVRCLHSVGAPLEPGQEDVAWPCNDTKYITQFPETKEIWSYGSGYGGNAILAKKCYALRIASVMAREEGWMAEHMLILKLINPEGKAYHIAAAFPSACGKTNLAMITPTIPGWTAQVVGDDIAWLKLREDGLYAVNPENGFFGVAPGTNYASNPIAMKTMEPGNTLFTNVALTDDGDIWWEGMDGDAPAHLIDWMGNDWTPESDENAAHPNSRYCVAIDQSPAAAPEFNDWEGVKIDAILFGGRRADTVPLVTQTYDWEHGTMVGALLASGQTAASAEAKVGTLRHDPMAMLPFIGYNAGEYLQNWIDMGNKGGDKMPSIFLVNWFRRGEDGRFLWPGFGDNSRVLKWVIDRIEGHVGADETVVGHTAKAEDLDLDGLDTPIEDVKEALTAPAEQWANDVEDNAEYLTFLGPRVPAEVHSQFDALKARISAAHA

SEQ ID NO: 6 - protein, 1231 aa, Mycobacterium bovis
MANISSPFGQNEWLVEAMYRKFRDDPSSVDPSWHEFLVDYSPEPTSQPAAEPTRVTSPLVAERAAAAAPQAPPKPADTAAAGNGVVAALAAKTAVPPPAEGDEVAVLRGAAAAVVKNMSASLEVPTATSVRAVPAKLLIDNRIVINNQLKRTRGGKISFTHLLGYALVQAVKKFPNMNRHYTEVDGKPTAVTPAHTNLGLAIDLQGKDGKRSLVVAGIKRCETMRFAQFVTAYEDIVRRARDGKLTTEDFAGVTISLTNPGTIGTVHSVPRLMPGQGAIIGVGAMEYPAEFQGASEERIAELGIGKLITLTSTYDHRIIQGAESGDFLRTIHELLLSDGFWDEVFRELSIPYLPVRWSTDNPDSIVDKNARVMNLIAAYRNRGHLMADTDPLRLDKARFRSHPDLEVLTHGLTLWDLDRVFKVDGFAGAQYKKLRDVLGLLRDAYCRHIGVEYAHILDPEQKEWLEQRVETKHVKPTVAQQKYILSKLNAAEAFETFLQTKYVGQKRFSLEGAESVIPMMDAAIDQCAEHGLDEVVIGMPHRGRLNVLANIVGKPYSQIFTEFEGNLNPSQAHGSGDVKYHLGATGLYLQMFGDNDIQVSLTANPSHLEAVDPVLEGLVRAKQDLLDHGSIDSDGQRAFSVVPLMLHGDAAFAGQGVVAETLNLANLPGYRVGGTIHIIVNNQIGFTTAPEYSRSSEYCTDVAKMIGAPIFHVNGDDPEACVWVARLAVDFRQRFKKDVVIDMLCYRRRGHNEGDDPSMTNPYMYDVVDTKRGARKSYTEALIGRGDISMKEAEDALRDYQGQLERVFNEVRELEKHGVQPSESVESDQMIPAGLATAVDKSLLARIGDAFLALPNGFTAHPRVQPVLEKRREMAYEGKIDWAFGELLALGSLVAEGKLVRLSGQDSRRGTFSQRHSVLIDRHTGEEFTPLQLLATNSDGSPTGGKFLVYDSPLSEYAAVGFEYGYTVGNPDAVVLWEAQFGDFVNGAQSIIDEFISSGEAKWGQLSNVVLLLPHGHEGQGPDHTSARIERFLQLWAEGSMTIAMPSTPSNYFHLLRRHALDGIQRPLIVFTPKSMLRHKAAVSEIKDFTEIKFRSVLEEPTYEDGIGDRNKVSRILLTSGKLYYELAARKAKDNRNDLAIVRLEQLAPLPRRRLRETLDRYENVKEFFWVQEEPANQGAWPRFGLELPELLPDKLAGIKRISRRAMSAPSSGSSKVHAVEQQEILDEAFG

SEQ ID NO: 7 - protein, 314 aa, Corynebacterium glutamicum
MKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 8 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 9 - protein, 461 aa, Corynebacterium glutamicum
MSDTPTSALITTVNRSFDGFDLEEVAADLGVRLTYLPDEELEVSKVLAADLLAEGPALIIGVGNTFFDAQVAAALGVPVLLLVDKQGKHVALARTQVNNAGAVVAAAFTAEQEPMPDKLRKAVRNHSNLEPVMSAELFENWLLKRARAEHSHIVLPEGDDDRILMAAHQLLDQDICDITILGDPVKIKERATELGLHLNTAYLVNPLTDPRLEEFAEQFAELRKSKSVTIDEAREIMKDISYFGTMMVHNGDADGMVSGAANTTAHTIKPSFQIIKTVPEASVVSSIFLMVLRGRLWAFGDCAVNPNPTAEQLGEIAVVSAKTAAQFGIDPRVAILSYSTGNSGGGSDVDRAIDALAEARRLNPELCVDGPLQFDAAVDPGVARKKMPDSDVAGQANVFIFPDLEAGNIGYKTAQRTGHALAVGPILQGLNKPVNDLSRGATVPDIVNTVAITAIQAGGRS

SEQ ID NO: 10 - protein, 397 aa, Corynebacterium glutamicum
MALALVLNSGSSSIKFQLVNPENSAIDEPYVSGLVEQIGEPNGRIVLKIEGEKYTLETPIADHSEGLNLAFDLMDQHNCGPSQLEITAVGHRVVHGGILFSAPELITDEIVEMIRDLIPLAPLHNPANVDGIDVARKILPDVPHVAVFDTGFFHSLPPAAALYAINKDVAAEHGIRRYGFHGTSHEFVSKRVVEILEKPTEDINTITFHLGNGASMAAVQGGRAVDTSMGMTPLAGLVMGTRSGDIDPGIVFHLSRTAGMSIDEIDNLLNKKSGVKGLSGVNDFRELREMIDNNDQDAWSAYNIYIHQLRRYLGSYMVALGRVDTIVFTAGVGENAQFVREDALAGLEMYGIEIDPERNALPNDGPRLISTDASKVKVFVIPTNEELAIARYAVKFA

SEQ ID NO: 11 - protein, 250 aa, Corynebacterium glutamicum
MSHMINKSISSTAEAVADIPDGASIAVGGFGLVGIPTALILALREQGAGDLTIISNNLGTDGFGLGLLLLDKKISKSIGSYLGSNKEYARQYLEGELTVEFTPQGTLAERLRAGGAGIPAFYTTAGVGTQVAEGGLPQRYNTDGTVAVVSQPKETREFNGQLYVMEEGIRADYALVHAHKADRFGNLVFRKTAQNFNPDAAMSGKITIAQVEHFVDELHPDEIDLPGIYVNRVVHVGPQETGIENRTVSN

SEQ ID NO: 12 - protein, 1140 aa, Corynebacterium glutamicum
MSTHTSSTLPAFKKILVANRGEIAVRAFRAALETGAATVAIYPREDRGSFHRSFASEAVRIGTEGSPVKAYLDIDEIIGAAKKVKADAIYPGYGFLSENAQLARECAENGITFIGPTPEVLDLTGDKSRAVTAAKKAGLPVLAESTPSKNIDEIVKSAEGQTYPIFVKAVAGGGGRGMRFVASPDELRKLATEASREAEAAFGDGAVYVERAVINPQHIEVQILGDHTGEVVHLYERDCSLQRRHQKVVEIAPAQHLDPELRDRICADAVKFCRSIGYQGAGTVEFLVDEKGNHVFIEMNPRIQVEHTVTEEVTEVDLVKAQMRLAAGATLKELGLTQDKIKTHGAALQCRITTEDPNNGFRPDTGTITAYRSPGGAGVRLDGAAQLGGEITAHFDSMLVKMTCRGSDFETAVARAQRALAEFTVSGVATNIGFLRALLREEDFTSKRIATGFIADHPHLLQAPPADDEQGRILDYLADVTVNKPHGVRPKDVAAPIDKLPNIKDLPLPRGSRDRLKQLGPAAFARDLREQDALAVTDTTFRDAHQSLLATRVRSFALKPAAEAVAKLTPELLSVEAWGGATYDVAMRFLFEDPWDRLDELREAMPNVNIQMLLRGRNTVGYTPYPDSVCRAFVKEAASSGVDIFRIFDALNDVSQMRPAIDAVLETNTAVAEVAMAYSGDLSDPNEKLYTLDYYLKMAEEIVKSGAHILAIKDMAGLLRPAAVTKLVTALRREFDLPVHVHTHDTAGGQLATYFAAAQAGADAVDGASAPLSGTTSQPSLSAIVAAFAHTRRDTGLSLEAVSDLEPYWEAVRGLYLPFESGTPGPTGRVYRHEIPGGQLSNLRAQATALGLADRFELIEDNYAAVNEMLGRPTKVTPSSKVVGDLALHLVGAGVDPADFAADPQKYDIPDSVIAFLRGELGNPPGGWPEPLRTRALEGRSEGKAPLTEVPEEEQAHLDADDSKERRNSLNRLLFPKPTEEFLEHRRRFGNTSALDDREFFYGLVEGRETLIRLPDVRTPLLVRLDAISEPDDKGMRNVVANVNGQIRPMRVRDRSVESVTATAEKADSSNKGHVAAPFAGVVTVTVAEGDEVKAGDAVAIIEAMKMEATITASVDGKIDRVVVPAATKVEGGDLIVVVS

SEQ ID NO: 13 - DNA, 43 bp, Artificial Sequence
  Synthetic ldhA_5′_HindIII
catgattacgccaagcttgagagcccaccacattgcgatttcc

SEQ ID NO: 14 - DNA, 42 bp, Artificial Sequence
  Synthetic ldhA_up_3′_XhoI
tcgaaactcgagtttcgatcccacttcctgatttccctaacc

SEQ ID NO: 15 - DNA, 39 bp, Artificial Sequence
  Synthetic ldhA_dn_5′_XhoI
tcgaaactcgagtaaatctttggcgcctagttggcgacg

SEQ ID NO: 16 - DNA, 46 bp, Artificial Sequence
  Synthetic ldhA_3′_EcoRI
acgacggccagtgaattcgacgacatctgagggtggataaagtggg

SEQ ID NO: 17 - DNA, 42 bp, Artificial Sequence
  Synthetic poxB 5′ H3
catgattacgccaagctttcagcgtgggtcgggttctttgag

SEQ ID NO: 18 - DNA, 32 bp, Artificial Sequence
  Synthetic DpoxB_up 3′
aatcatcatctgaactcctcaacgttatggct

SEQ ID NO: 19 - DNA, 37 bp, Artificial Sequence
  Synthetic DpoxB_dn 5′
ggagttcagatgatgattgatacacctgctgttctca

SEQ ID NO: 20 - DNA, 44 bp, Artificial Sequence
  Synthetic poxB 3′ E1
acgacggccagtgaattcatgtcccgaatccacttcaatcagag

SEQ ID NO: 21 - DNA, 44 bp, Artificial Sequence
  Synthetic pta 5′ H3
catgattacgccaagcttccctccatgatacgtggtaagtgcag

SEQ ID NO: 22 - DNA, 43 bp, Artificial Sequence
  Synthetic Dpta_up_R1 3′
gttccctgttaatgtaaccagctgaggtcggtgtgtcagacat

SEQ ID NO: 23 - DNA, 48 bp, Artificial Sequence
  Synthetic DackA_dn_R1 5′
ttacattaacagggaaccggaagagttagctatcgctaggtacgcggt

SEQ ID NO: 24 - DNA, 40 bp, Artificial Sequence
  Synthetic ackA 3′ Xb
acccggggatcctctagagggctgatgtgatttctgcggg

SEQ ID NO: 25 - DNA, 41 bp, Artificial Sequence
  Synthetic actA 5′ Xb
ggtggcggccgctctagaggtctgagctttattcctgggct

SEQ ID NO: 26 - DNA, 36 bp, Artificial Sequence
  Synthetic DactA_up_R4 3′
tctggatagaagcatctaagccagcgccggtgaagc

SEQ ID NO: 27 - DNA, 46 bp, Artificial Sequence
  Synthetic DactA_dn_R4 5′
agatgcttctatccagagctccggtgacaacaagtacatgcagacc

SEQ ID NO: 28 - DNA, 39 bp, Artificial Sequence
  Synthetic actA 3′ H3
gacggtatcgataagcttcgtacgatgcttgagcggtat

SEQ ID NO: 29 - DNA, 19 bp, Artificial Sequence
  Synthetic poxB_up_for
ggctgaaaccaaaccagac

SEQ ID NO: 30 - DNA, 22 bp, Artificial Sequence
  Synthetic poxB_dn_rev
ctgcatgatcggttagatacag

SEQ ID NO: 31 - DNA, 18 bp, Artificial Sequence
  Synthetic pta_up_for
gcgtggaattgagatcgg

SEQ ID NO: 32 - DNA, 18 bp, Artificial Sequence
  Synthetic ackA_dn_rev
cagagcgatttgtggtgg

SEQ ID NO: 33 - DNA, 20 bp, Artificial Sequence
  Synthetic actA_up_for
tgaagcaatggtgtgaactg

SEQ ID NO: 34 - DNA, 19 bp, Artificial Sequence
  Synthetic actA_dn_rev
gctaccaaacactagcctg

SEQ ID NO: 35 - DNA, 25 bp, Artificial Sequence
  Synthetic pyc-F1
gctctagattgagcacaccgtgact

SEQ ID NO: 36 - DNA, 19 bp, Artificial Sequence
  Synthetic pyc-R1
ctgaaggaggtgcgagtga

SEQ ID NO: 37 - DNA, 19 bp, Artificial Sequence
  Synthetic pyc-F2
tcactcgcacctccttcag

SEQ ID NO: 38 - DNA, 31 bp, Artificial Sequence
  Synthetic pyc-R2
gctctagagaagcagcatctgaatgtttaca

SEQ ID NO: 39 - DNA, 46 bp, Artificial Sequence
  Synthetic MD-616
aaagtgtaaagcctgggaacaacaagacccatcatagtttgccccc

SEQ ID NO: 40 - DNA, 36 bp, Artificial Sequence
  Synthetic MD-618
gttcttctaatcagaattggttaattggttgtaaca

SEQ ID NO: 41 - DNA, 40 bp, Artificial Sequence
  Synthetic MD-615
gcgtaatagcgaagaggggcgtttttccataggctccgcc

SEQ ID NO: 42 - DNA, 40 bp, Artificial Sequence
  Synthetic MD-617
gttcaatcataacaccccttgtattactgtttatgtaagc

SEQ ID NO: 43 - DNA, 31 bp, Artificial Sequence
  Synthetic MD-619
gggtgttatgattgaacaagatggattgcac

SEQ ID NO: 44 - DNA, 39 bp, Artificial Sequence
  Synthetic MD-620
attctgattagaagaactcgtcaagaaggcgatagaagg

SEQ ID NO: 45 - DNA, 17 bp, Artificial Sequence
  Synthetic LacZa-NR
cctcttcgctattacgc

SEQ ID NO: 46 - DNA, 21 bp, Artificial Sequence
  MD-404
cccaggctttacactttatgc

SEQ ID NO: 47 - DNA, 47 bp, Artificial Sequence
  Synthetic MD-627
gccaccgcggtggagctcatttagcggatgattctcgttcaacttcg

SEQ ID NO: 48 - DNA, 32 bp, Artificial Sequence
  Synthetic MD-628
ttttatttgcaaaaacggccgaaaccatccct

SEQ ID NO: 49 - DNA, 40 bp, Artificial Sequence
  Synthetic MD-629
ccgtttttgcaaataaaacgaaaggctcagtcgaaagact

SEQ ID NO: 50 - DNA, 43 bp, Artificial Sequence
  Synthetic MD-630
gaacaaaagctggagctaccgtatctgtggggggatggcttgt

SEQ ID NO: 51 - DNA, 50 bp, Artificial Sequence
  Synthetic MD-1452
ctatagggcgaattggtgcgttaataaaggtggagaataagttgtttcca

SEQ ID NO: 52 - DNA, 55 bp, Artificial Sequence
  Synthetic MD-1453
ggtaccgttgtcctctcttatcgagtttagattccctaaacttttatcgaggtaa

SEQ ID NO: 53 - DNA, 40 bp, Artificial Sequence
  Synthetic MD-1557
cgataagagaggacaacgatggtgagcaagggcgaggagc

SEQ ID NO: 54 - DNA, 42 bp, Artificial Sequence
  Synthetic MD-1620
agaatcatccgctaaatgatctcagtggtggtggtggtggtg

SEQ ID NO: 55 - DNA, 41 bp, Artificial Sequence
  Synthetic MD-1357
caaatgggtcgcggatccatggtgagcaagggcgaggagct

SEQ ID NO: 56 - DNA, 35 bp, Artificial Sequence
  Synthetic MD-1358
gtggtggtggtgctcgaccttgtacagctcgtcca

SEQ ID NO: 57 - DNA, 753 bp, Artificial Sequence
  Synthetic 753
caaatgggtcgcggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggtcgagcaccaccaccac

SEQ ID NO: 58 - DNA, 6052 bp, Artificial Sequence
  Synthetic MD0545
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgaccttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggatccgcgacccatttgctgtccaccagtcatgctagccatatggctgccgcgcggcaccaggccgctgctgtgatgatgatgatgatggctgctgcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca